SEQUENCE LISTING (58 sequences)

SEQ ID NO: 1 - protein, 773 aa, Homo sapiens
METDESPSPLPCGPAGEAVMESRARPFQALPREQSPPPPLQTSSGAEVMDVGSGGDGQSELPAEDPFNFYGASLLSKGSFSKGRLLIDPNCSGHSPRTARHAPAVRKFSPDLKLLKDVKISVSFTESCRSKDRKVLYTGAERDVRAECGLLLSPVSGDVHACPFGGSVGDGVGIGGESADKKDEENELDQEKRVEYAVLDELEDFTDNLELDEEGAGGFTAKAIVQRDRVDEEALNFPYEDDFDNDVDALLEEGLCAPKKRRTEEKYGGDSDHPSDGETSVQPMMTKIKTVLKSRGRPPTEPLPDGWIMTFHNSGVPVYLHRESRVVTWSRPYFLGTGSIRKHDPPLSSIPCLHYKKMKDNEEREQSSDLTPSGDVSPVKPLSRSAELEFPLDEPDSMGADPGPPDEKDPLGAEAAPGALGQVKAKVEVCKDESVDLEEFRSYLEKRFDFEQVTVKKFRTWAERRQFNREMKRKQAESERPILPANQKLITLSVQDAPTKKEFVINPNGKSEVCILHEYMQRVLKVRPVYNFFECENPSEPFGASVTIDGVTYGSGTASSKKLAKNKAARATLEILIPDFVKQTSEEKPKDSEELEYFNHISIEDSRVYELTSKAGLLSPYQILHECLKRNHGMGDTSIKFEVVPGKNQKSEYVMACGKHTVRGWCKNKRVGKQLASQKILQLLHPHVKNWGSLLRMYGRESSKMVKQETSDKSVIELQQYAKKNKPNLHILSKLQEEMKRLAEEREETRKKPKMSIVASAQPGGEPLCTVDV

SEQ ID NO: 2 - protein, 490 aa, Homo sapiens
MMTKIKTVLKSRGRPPTEPLPDGWIMTFHNSGVPVYLHRESRVVTWSRPYFLGTGSIRKHDPPLSSIPCLHYKKMKDNEEREQSSDLTPSGDVSPVKPLSRSAELEFPLDEPDSMGADPGPPDEKDPLGAEAAPGALGQVKAKVEVCKDESVDLEEFRSYLEKRFDFEQVTVKKFRTWAERRQFNREMKRKQAESERPILPANQKLITLSVQDAPTKKEFVINPNGKSEVCILHEYMQRVLKVRPVYNFFECENPSEPFGASVTIDGVTYGSGTASSKKLAKNKAARATLEILIPDFVKQTSEEKPKDSEELEYFNHISIEDSRVYELTSKAGLLSPYQILHECLKRNHGMGDTSIKFEVVPGKNQKSEYVMACGKHTVRGWCKNKRVGKQLASQKILQLLHPHVKNWGSLLRMYGRESSKMVKQETSDKSVIELQQYAKKNKPNLHILSKLQEEMKRLAEEREETRKKPKMSIVASAQPGGEPLCTVDV

SEQ ID NO: 3 - protein, 247 aa, Homo sapiens
METDESPSPLPCGPAGEAVMESRARPFQALPREQSPPPPLQTSSGAEVMDVGSGGDGQSELPAEDPFNFYGASLLSKGSFSKGRLLIDPNCSGHSPRTARHAPAVRKFSPDLKLLKDVKISVSFTESCRSKDRKVLYTGAERDVRAECGLLLSPVSGDVHACPFGGSVGDGVGIGGESADKKDEENELDQEKRVEYAVLDELEDFTDNLELDEEGAGGFTAKAIVQRDRVDEEALNFPYEVCWQPLL

SEQ ID NO: 4 - protein, 773 aa, Homo sapiens
METYESPSPLPREPAGEAMMENRACPFQVLPHEQSPPPPLQTSSDAEVMDVGSGGDGQSEPPADDPFNFYGASLLSKGSFSKGRLLIDPNCSGHSPRTARHAPAVRKFSPDLKLLKDVKISVSFTESCRSKDRKVLYTGVERSTRPECGQLLSPVSGDVHACPFGGSVGNGVGLGGESADKKDEENELDQEKRVEYAVLDELEDFTDNLELDEEGTGGFTAKAIVQRDRVDEEALNFSYEDDFDNDVDALLEEGLCAPKKRRMEEKYGGDSDHPSDGETSVQPMMTKIKTVLKSRGRPPTEPLPDGWIMTFHNSGVPVYLHRESRVVTWSRPYFLGTGSIRKHDPPLSSIPCLHYKKMKDNEEREQNCDLAPSGEVSPVKPLGRSAELDFPLEEPDSMGGDSGSMDEKDPLGAEAAAGALGQVKAKVEVCKDESVDLEEFRNYLEKRFDFEQVTVKKFRTWAERRQFNREMKRKQAESERPILPANQKLITLSVQDAPTKKEFVINPNGKSEVCILHEYMQRVLKVRPVYNFFECENPSEPFGASVTIDGVTYGSGTASSKKLAKNKAARATLEILIPDFVKQTSEEKPKDSEELEYFNHISIEDSRVYELTSKAGLLSPYQILHECLKRNHGMGDTSIKFEVVPGKNQKSEYVMACGKHTVRGWCKNKRVGKQLASQKILQLLHPHVKNWGSLLRMYGRESSKMVKQETSDKSVIELQQYAKKNRPNLHILSKLQEEMKRLAAEREETRKKPKMSIVASAQPGGEPLCTVDV

SEQ ID NO: 5 - protein, 773 aa, Homo sapiens; identical to SEQ ID NO: 4

SEQ ID NO: 6 - protein, 773 aa, Homo sapiens; identical to SEQ ID NO: 4

SEQ ID NO: 7 - protein, 611 aa, Homo sapiens
METYESPSPLPREPAGEAMMENRACPFQVLPHEQSPPPPLQTSSDAEVMDVGSGGDGQSEPPADDPFNFYGASLLSKGSFSKGRLLIDPNCSGHSPRTARHAPAVRKFSPDLKLLKDVKISVSFTESCRSKDRKVLYTGVERSTRPECGQLLSPVSGDVHACPFGGSVGNGVGLGGESADKKDEENELDQEKRVEYAVLDELEDFTDNLELDEEGTGGFTAKAIVQRDRVDEEALNFSYEDDFDNDVDALLEEGLCAPKKRRMEEKYGGDSDHPSDGETSVQPMMTKIKTVLKSRGRPPTEPLPDGWIMTFHNSGVPVYLHRESRVVTWSRPYFLGTGSIRKHDPPLSSIPCLHYKKMKDNEEREQNCDLAPSGEVSPVKPLGRSAELDFPLEEPDSMGGDSGSMDEKDPLGAEAAAGALGQVKAKVEVCKDESVDLEEFRNYLEKRFDFEQVTVKKFRTWAERRQFNREMKRKQAESERPILPANQKLITLSVQDAPTKKEFVINPNGKSEVCILHEYMQRVLKVRPVYNFFECENPSEPFGASVTIDGVTYGSGTASSKKLAKNKAARATLEILIPDFVKQTSEEKPKDSEELEVSVVILFSWELPYSH

SEQ ID NO: 8 - protein, 588 aa, Homo sapiens
FHLSFQPVCFSDDQMPSGHAQSPLRGSSVCSLIHVKLVLGAKAWRGVGSEHFILQDDFDNDVDALLEEGLCAPKKRRMEEKYGGDSDHPSDGETSVQPMMTKIKTVLKSRGRPPTEPLPDGWIMTFHNSGVPVYLHRESRVVTWSRPYFLGTGSIRKHDPPLSSIPCLHYKKMKDNEEREQNCDLAPSGEVSPVKPLGRSAELDFPLEEPDSMGGDSGSMDEKDPLGAEAAAGALGQVKAKVEVCKDESVDLEEFRNYLEKRFDFEQVTVKKFRTWAERRQFNREMKRKQAESERPILPANQKLITLSVQDAPTKKEFVINPNGKSEVCILHEYMQRVLKVRPVYNFFECENPSEPFGASVTIDGVTYGSGTASSKKLAKNKAARATLEILIPDFVKQTSEEKPKDSEELEYFNHISIEDSRVYELTSKAGLLSPYQILHECLKRNHGMGDTSIKFEVVPGKNQKSEYVMACGKHTVRGWCKNKRVGKQLASQKILQLLHPHVKNWGSLLRMYGRESSKMVKQETSDKSVIELQQYAKKNRPNLHILSKLQEEMKRLAAEREETRKKPKMSIVASAQPGGEPLCTVDV

SEQ ID NO: 9 - protein, 773 aa, Homo sapiens
METYESPSPLPHEPTGEAMMENRACPFQVLPHEQSPPPPLQTSSDAEVMDVGSGGDGQSEPPADDPFNFYGASLLSKGSFSKGRLLIDPNCSGHSPRTARHAPAVRKFSPDLKLLKDVKISVSFTESCRSKDRKVLYTGVERSTRPECSQLLSPVCGDVHACPFGGSVGNGVGLGGESTDKKDEENELDQEKRVEYAVLDELEDFTDNLELDEEGTGGFTANAIVQRDRVDEEALNFSYEDDFDNDVDALLEEGLCAPKKRRMEEKYGGDSDHPSDGETSVQPMMTKIKTVLKSRGRPPTEPLPDGWIMTFHNSGVPVYLHRESRVVTWSRPYFLGTGSIRKHDPPLSSIPCLHYKKMKDNEEREQSCDLAPSGEVSPVKPLGRSAELDFPLEEPDSLGGDSGSLDEKDPLGAEAAAGALGQVRAKVEVCKDESVDLEEFRNYLEKRFDFEQVTVKKFRTWAERRQFNREMKRKQAESERPILPANQKLITLSVQDAPTKKEFVINPNGKSEVCILHEYMQRVLKVRPVYNFFECENPSEPFGASVTIDGVTYGSGTASSKKLAKNKAARATLEILIPDFVKQTSEEKPKDSEELEYFNHISIEDSRVYELTSKAGLLSPYQILHECLKRNHGMGDTSIKFEVVPGKNQKSEYVMACGKHTVRGWCKNKRVGKQLASQKILQLLHPHVKNWGSLLRMYGRESSKMVKQETSDKSVIELQQYAKKNRPNLHILSKLQEEMKRLAAEREETRKKPKMSIVASAQPGGEPLCTVDV

SEQ ID NO: 10 - protein, 773 aa, Homo sapiens; identical to SEQ ID NO: 9

SEQ ID NO: 11 - protein, 620 aa, Homo sapiens
METYESPSPLPHEPTGEAMMENRACPFQVLPHEQSPPPPLQTSSDAEVMDVGSGGDGQSEPPADDPFNFYGASLLSKGSFSKGRLLIDPNCSGHSPRTARHAPAVRKFSPDLKLLKDVKISVSFTESCRSKDRKVLYTGVERSTRPECSQLLSPVCGDVHACPFGGSVGNGVGLGGESTDKKDEENELDQEKRVEYAVLDELEDFTDNLELDEEGTGGFTANAIVQRDRVDEEALNFSYEDDFDNDVDALLEEGLCAPKKRRMEEKYGGDSDHPSDGETSVQPMMTKIKTVLKSRGRPPTEPLPDGWIMTFHNSGVPVYLHRESRVVTWSRPYFLGTGSIRKHDPPLSSIPCLHYKKMKDNEEREQSCDLAPSGEVSPVKPLGRSAELDFPLEEPDSLGGDSGSLDEKDPLGAEAAAGALGQVRAKVEVCKDESVDLEEFRNYLEKRFDFEQVTVKKFRTWAERRQFNREMKRKQAESERPILPANQKLITLSVQDAPTKKEFVINPNGKSEVCILHEYMQRVLKVRPVYNFFECENPSEPFGASVTIDGVTYGSGTASSKKLAKNKAARATLEILIPDFVKQTSEEKPKDSEELEVSVGILFSWELHLPYIASTRLRTM

SEQ ID NO: 12 - DNA, 8448 bp, Artificial
  nucleic sequence coding for the fusion protein DGCR8-GFP
gacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggcttatcgaaattaatacgactcactatagggagacccaagcttggtaccgagctcggatcgatccatcgatagtgtggtggaattctgatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggaattctgatcgccgccgccatggactacaaggacgacgatgacaagtacccttatgacgtgcccgattacgctttgaaccagatatcaacaagtttgtacaaaaaagcaggctccaccatgggaaccaattcagtcgacatggagacagatgagagcccctctccgctcccgtgtgggcccgcaggagaagcggtgatggagagccgagctcgccccttccaagcgctgccccgtgagcagtctccaccacctcccctgcaaacgtccagtggtgcagaggtaatggacgttggctctggtggtgatggacagtccgaactccctgctgaggaccccttcaacttctacggagcttctcttctctccaaaggatccttctctaagggccgcctcctcatagacccgaactgtagtggccacagcccgcgcaccgcccggcacgcacctgcggtccggaagttctcccctgaccttaagttgcttaaggatgtaaagattagcgtgagctttaccgagagctgcaggagtaaggacaggaaggtgctgtacacaggagcagagcgcgacgtgcgggcggagtgcggtctgctccttagccctgtcagtggggacgtgcatgcttgtccctttggcgggagtgttggtgacggggtaggcatagggggtgagagtgctgataagaaggatgaggagaatgagctggatcaggaaaagagagtggagtatgcagtgctcgatgagttagaagattttactgacaatttggagctagatgaagaaggagcaggcgggttcacggctaaagcaatcgttcagagagacagagtggatgaagaggccttgaatttcccctacgaggatgactttgacaacgatgtggatgctctgctggaagaaggcctttgtgcccccaaaaagaggcgaacagaggaaaaatatggcggagacagcgaccatccgtccgatggagagacaagtgtgcagccgatgatgaccaagattaaaacagtgctcaaaagtcgtggccgcccacctacagagccgctgcccgacgggtggatcatgacattccataactctggagtcccggtgtacctacacagagagtctcgggtggtcacctggtccaggccatacttcttgggaacgggaagcatacggaaacacgaccctcctctgagtagcatcccttgtctgcattataagaaaatgaaggacaacgaggaacgggagcaaagcagtgacctcacccctagtggggatgtgtcccccgtcaagcccctgagccgatctgcagagctggagtttcccctggatgagcctgactctatgggtgctgacccggggcccccggacgagaaagacccactaggggctgaggcagcccctggggccctggggcaggtgaaggccaaagtcgaggtgtgcaaagatgaatccgttgatctcgaggaatttcgaagctacctggagaagcgttttgactttgagcaagttactgtgaaaaaattcaggacttgggctgagcggcggcaattcaatcgggaaatgaagcggaagcaggcggagtccgagaggcccatcttgccagccaatcagaagctcattactttatcagtgcaagatgcacccacaaagaaagagtttgttattaaccccaacgggaaatccgaggtctgcatcctgcacgagtacatgcagcgtgtcctcaaggtccgccctgtctataatttctttgaatgtgagaacccaagtgagccttttggtgcctcggtgaccattgatggtgtgacttacggatctggaactgcaagcagcaaaaaacttgcgaagaataaagctgcccgagctacactggaaatcctcatccctgactttgttaaacagacctctgaagagaagcccaaagacagtgaagaactcgagtattttaaccacatcagcatcgaggactcgcgggtctacgagctgaccagcaaggctgggctgttgtctccatatcagatcctccacgagtgccttaaaagaaaccatgggatgggtgacacgtctatcaagtttgaagtggttcctgggaaaaaccagaagagtgaatacgtcatggcgtgtggcaagcacacagtgcgcgggtggtgtaagaacaagagagttggaaagcagttagcctcacagaagatccttcagctgctgcacccacatgtcaagaactgggggtctttactgcgcatgtatggccgtgagagcagcaagatggtcaagcaggagacatcggacaagagtgtgattgagctgcagcagtatgccaagaagaacaagcccaacctgcacatcctcagcaagctccaagaggagatgaagaggctagctgaggaaagggaggagactcgaaagaagcccaagatgtccattgtggcgtccgcccagcctggcggtgagcccctgtgcaccgtggacgtgtgagcggccgcactcgagatatctagacccagctttcttgtacaaagtggttgatatccagcacagtggcggccgctcaagtctagagggcccgcggttcgaaggtaagcctatccctaaccctctcctcggtctcgattctacgcgtacctgatccgcggccgcatagataactgatccagtgtgctggaattaattcgctgtctgcgagggccagctgttggggtgagtactccctctcaaaagcgggcatgacttctgcgctaagattgtcagtttccaaaaacgaggaggatttgatattcacctggcccgcggtgatgcctttgagggtggccgcgtccatctggtcagaaaagacaatctttttgttgtcaagcttgaggtgtggcaggcttgagatctggccatacacttgagtgacaatgacatccactttgcctttctctccacaggtgtccactcccaggtccaactgcaggtcgagcatgcatctagggcggccaattccgcccctctccctcccccccccctaacgttactggccgaagccgcttggaataaggccggtgtgcgtttgtctatatgtgattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataagcttgccacaacccacaaggagacgaccttccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgaccccatggctccgaccgaagccgacccgggcggccccgccgaccccgcacccgcccccgaggcccaccgactctagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtcgagtgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 13 - RNA, 84 nt, Homo sapiens
ucucagucuguggcacucagccuugagggcacuuucuggugccagaaugaaagugcugucauagcugagguccaaugacugagg

SEQ ID NO: 14 - RNA, 98 nt, Homo sapiens
gguacuucucagucuguggcacucagccuugagggcacuuucuggugccagaaugaaagugcugucauagcugagguccaaugacugaggcgagcacc

SEQ ID NO: 15 - RNA, 73 nt, Homo sapiens
acugagguccucaaaacugaggggcauuuucugugguuugaaaggaaagugcacccaguuuuggggaugucaa

SEQ ID NO: 16 - RNA, 124 nt, Homo sapiens
aacccuccuugggaagugaagcucaggcugugauuucaagccagggggcguuuuucuauaacuggaugaaaagcaccuccagagcuugaagcucacaguuugagagcaaucgucuaaggaaguu

SEQ ID NO: 17 - RNA, 87 nt, Homo sapiens
ucuccugcugugacccucaagauggaagcaguuucuguugucugaaaggaaagaaagugcuuccuuuuugaggguuacuguuugaga

SEQ ID NO: 18 - RNA, 83 nt, Homo sapiens
ucucaugcagucauucuccaaaagaaagcacuuucuguugucugaaagcagagugccuucuuuuggagcguuacuguuugaga

SEQ ID NO: 19 - RNA, 84 nt, Homo sapiens
ucucaugcagucauucuccaaaagggagcacuuucuguuugaaagaaaacaaagugccuccuuuuagaguguuacuguuugaga

SEQ ID NO: 20 - RNA, 87 nt, Homo sapiens
ucucaggcugugacccucuaaagggaagcgcuuucuguggucagaaagaaaagcaagugcuuccuuuuagaggguuaccguuuggga

SEQ ID NO: 21 - RNA, 83 nt, Homo sapiens; identical to SEQ ID NO: 18

SEQ ID NO: 22 - RNA, 87 nt, Homo sapiens
ucucagccugugacccucuagagggaagcgcuuucuguugucugaaagaaaagaaagugcaucuuuuuagaggauuacaguuugaga

SEQ ID NO: 23 - RNA, 87 nt, Homo sapiens
cucaagcuaugagucuacaaaggaaagcgcuuucuguugucagaaagaagagaaagcgcuucccuuuugaggguuacgguuugagaa

SEQ ID NO: 24 - RNA, 85 nt, Homo sapiens
cucaggcugugacccuccagagggaaguacuuucuguugucugagagaaaagaaagugcuucccuuuggacuguuucgguuugag

SEQ ID NO: 25 - RNA, 83 nt, Homo sapiens
ucaggcugugacccucuugagggaagcacuuucuguugucugaaagaagagaaagugcuuccuuuuagaggcuuacugucuga

SEQ ID NO: 26 - RNA, 81 nt, Homo sapiens
caugcugugacccucuagagggaagcgcuuucuguugucugaaagaaaagaaagugcauccuuuuagagguuuacuguuug

SEQ ID NO: 27 - RNA, 85 nt, Homo sapiens
cucaagcugugacucuccagagggaugcacuuucucuuaugugaaaaaaaagaaggcgcuucccuuuagagcguuacgguuuggg

SEQ ID NO: 28 - RNA, 87 nt, Homo sapiens
ucucaugcugugacccucuagagggaagcgcuuucuguugucugaaagaaaagaacgcgcuucccuauagaggguuacccuuugaga

SEQ ID NO: 29 - RNA, 87 nt, Homo sapiens
ucucaugcugugacccucuagagggaagcacuuucucuugucuaaaagaaaagaaagcgcuucucuuuagaggauuacucuuugaga

SEQ ID NO: 30 - RNA, 61 nt, Homo sapiens
cccucuacagggaagcgcuuucuguugucugaaagaaaagaaagugcuuccuuuuagaggg

SEQ ID NO: 31 - RNA, 83 nt, Homo sapiens
ucaugcuguggcccuccagagggaagcgcuuucuguugucugaaagaaaacaaagcgcuccccuuuagagguuuacgguuuga

SEQ ID NO: 32 - RNA, 85 nt, Homo sapiens
cucaggcugugacccucuagagggaagcacuuucuguugcuugaaagaagagaaagcgcuuccuuuuagaggauuacucuuugag

SEQ ID NO: 33 - RNA, 87 nt, Homo sapiens
ucucaggcugucguccucuagagggaagcacuuucuguugucugaaagaaaagaaagugcuuccuuuuagaggguuaccguuugaga

SEQ ID NO: 34 - RNA, 101 nt, Homo sapiens
gcgagaagaucucaugcugugacucucuggagggaagcacuuucuguugucugaaagaaaacaaagcgcuucucuuuagaguguuacgguuugagaaaagc

SEQ ID NO: 35 - RNA, 87 nt, Homo sapiens
ucucaugcugugacccuacaaagggaagcacuuucucuuguccaaaggaaaagaaggcgcuucccuuuggaguguuacgguuugaga

SEQ ID NO: 36 - RNA, 87 nt, Homo sapiens
ucucaggcagugacccucuagauggaagcacugucuguuguauaaaagaaaagaucgugcaucccuuuagaguguuacuguuugaga

SEQ ID NO: 37 - RNA, 88 nt, Homo sapiens
ucccaugcugugacccuccaaagggaagcgcuuucuguuuguuuucucuuaaacaaagugccucccuuuagaguguuaccguuuggga

SEQ ID NO: 38 - RNA, 87 nt, Homo sapiens
ucucgggcugugacucuccaaagggaagaauuuucucuugucuaaaagaaaagaacgcacuucccuuuagaguguuaccgugugaga

SEQ ID NO: 39 - RNA, 87 nt, Homo sapiens
ucucaagcugugagucuacaaagggaagcccuuucuguugucuaaaagaaaagaaagugcuucucuuugguggguuacgguuugaga

SEQ ID NO: 40 - RNA, 67 nt, Homo sapiens
gugacccucuagauggaagcacugucuguugucuaagaaaagaucgugcaucccuuuagaguguuac

SEQ ID NO: 41 - RNA, 90 nt, Homo sapiens
ucccaugcugugacccucuagaggaagcacuuucuguuuguugucugagaaaaaacaaagugcuucccuuuagaguguuaccguuuggga

SEQ ID NO: 42 - RNA, 85 nt, Homo sapiens
ucucaugaugugaccaucuggagguaagaagcacuuuguguuuugugaaagaaagugcuuccuuucagaggguuacucuuugaga

SEQ ID NO: 43 - RNA, 65 nt, Homo sapiens
gugacccucuagagggaagcacuuucuguugaaagaaaagaacaugcauccuuucagaggguuac

SEQ ID NO: 44 - RNA, 88 nt, Homo sapiens
ucucaggcugugacccucuagagggaagcgcuuucuguuggcuaaaagaaaagaaagcgcuucccuucagaguguuaacgcuuugaga

SEQ ID NO: 45 - RNA, 85 nt, Homo sapiens
ucucaagcugugacugcaaagggaagcccuuucuguugucugaaagaagagaaagcgcuucccuuugcuggauuacgguuugaga

SEQ ID NO: 46 - RNA, 87 nt, Homo sapiens
ucccaugcugugacccucuagagggaagcacuuucuguugucugaaagaaaccaaagcgcuucccuuuggagcguuacgguuugaga

SEQ ID NO: 47 - RNA, 90 nt, Homo sapiens
ucucaggcugugaccaucuggagguaagaagcacuuucuguuuugugaaagaaaagaaagugcuuccuuucagaggguuacucuuugaga

SEQ ID NO: 48 - RNA, 87 nt, Homo sapiens
ucucaagcugugggucugcaaagggaagcccuuucuguugucuaaaagaagagaaagcgcuucccuuugcuggauuacgguuugaga

SEQ ID NO: 49 - RNA, 95 nt, Homo sapiens
gaagaucucaggcagugacccucuagauggaagcacugucuguugucuaagaaaagaucgugcauccuuuuagaguguuacuguuugagaaaauc

SEQ ID NO: 50 - RNA, 88 nt, Homo sapiens
ucccaugcugugacccucuagaggaagcacuuucuguuuguugucugagaaaaaacaaagugcuucccuuuagaguuacuguuuggga

SEQ ID NO: 51 - RNA, 87 nt, Homo sapiens
ucucaggcugugacccuccaaagggaagaacuuucuguugucuaaaagaaaagaacgcacuucccuuuagaguguuaccgugugaga

SEQ ID NO: 52 - RNA, 87 nt, Homo sapiens
ucucaggcugugucccucuagagggaagcgcuuucuguugucugaaagaaaagaaaaugguucccuuuagaguguuacgcuuugaga

SEQ ID NO: 53 - RNA, 85 nt, Homo sapiens
cucaggcugugacacucuagagggaagcgcuuucuguugucugaaagaaaggaaagugcauccuuuuagaguguuacuguuugag

SEQ ID NO: 54 - RNA, 85 nt, Homo sapiens
ucucaagcugugacugcaaagggaagcccuuucuguugucuaaaagaaaagaaagugcuucccuuuggugaauuacgguuugaga

SEQ ID NO: 55 - RNA, 90 nt, Homo sapiens
ucucaggcugugaccuucucgaggaaagaagcacuuucuguugucugaaagaaaagaaagugcuuccuuucagaggguuacgguuugaga

SEQ ID NO: 56 - RNA, 87 nt, Homo sapiens
cucaagcugugagucuacaaaggaaagcgcuuucuguugucugaaagaaaagaaaucgcuucccuuuggaguguuacgguuugagaa

SEQ ID NO: 57 - RNA, 90 nt, Homo sapiens
ucucagguugugaccuucucgaggaaagaagcacuuucuguugucugaaagaaaagaaagugcuuccuuucagaggguuacgguuugaga

SEQ ID NO: 58 - RNA, 87 nt, Homo sapiens
ucucaggcugugucccucuacagggaagcgcuuucuguugucugaaagaaaggaaagugcauccuuuuagaguguuacuguuugaga

(19) United States
(12) Patent Application Publication
(10) Pub. No.: US 2012/0202209 A1
(43) Pub. Date: Aug. 9, 2012

(54) CELL IMAGING METHOD FOR VIEWING MICRORNA BIOGENESIS IN THE CELLS

(75) Inventors: Jerome Cavaille, Ramonville St Agne (FR); Marie-Line Bortolin-Cavaille, Ramonville St Agne (FR); Clement Bellemer, Toulouse (FR)

(73) Assignee: Centre National de la Recherche Scientifique - CNRS, Paris 16 (FR)

(21) Appl. No.: 13/497,669

(22) PCT Filed: Sep. 21, 2010
(86) PCT No.: PCT/FR2010/051974

§ 371 Date: Mar. 22, 2012

(30) Foreign Application Priority Data
Sep. 22, 2009 (FR) 09 56522

(51) Int. Cl. G01N 33/53; G01N 21/64

(52) U.S. Cl. 435/6.11; 435/7.1; 435/29; 435/6.13

(57) ABSTRACT

The present invention relates to a method for viewing the biogenesis of at least one microRNA, preferably a microRNA group, in a living cell, characterised in that said method includes the following steps: transforming said cell by an encoding vector for a protein selected among the DGCR8 protein (DiGeorge syndrome critical region gene 8), the Drosha protein and derivatives thereof, said protein being coupled with a marker; expressing said protein coupled with said marker; and detecting said marker.

The present invention relates to a method of visualizing the biogenesis of at least one microRNA, preferably a group of microRNAs, in a cell, based on the transformation of said cell by a vector coding for a protein selected from the DGCR8

(DiGeorge syndrome critical region gene 8) protein, the Drosha protein, and derivatives thereof, coupled to a marker, and detection of said marker. The microRNAs (miRNAs) are small RNAs (preferably between 15 to 30 nucleotides, and more preferably between 19 and 23 nucleotides) capable of negatively modulating gene expression. Through their capacity for regulating a large number of genes, they are involved in many biological processes such as control of apoptosis, and of cellular proliferation and differentiation.

The production of microRNAs begins in the nucleus by cleavage of a precursor RNA: pri-miRNA. These cleavages are performed by a specific complex: “the Microprocessor” comprising the DGCR8 and Drosha proteins, said cleavages liberating a labile intermediate, pre-miRNA, from which the microRNA is generated as a result of a second series of cleavages catalyzed by the protein DICER.

Deregulations of the production of microRNAs, both transcriptional and posttranscriptional, are frequently described in pathological contexts such as cancers, suggesting that the microRNAs might constitute therapeutic targets in certain disorders.

Thus, it would be interesting to have a screening tool at our disposal, for identifying the molecules or cellular factors capable of modulating the production of microRNAs. The present invention relates to a cell imaging method making it possible to monitor, in living cells, the dynamics and the recruitment of the Microprocessor (Drosha-DGCR8 complex) on nascent transcripts. Using a cellular system of the reporter gene type, it is possible to screen molecules on living human cells with the main objective of identifying drugs or cellular factors capable of modulating, positively or negatively, the activity of the Microprocessor, and therefore the production of microRNAs. The inventors have identified a system for monitoring the dynamics and mode of action of the Microprocessor in cells, notably living human cells, more particularly in the cells of the line of human choriocarcinoma JEG3 (ATCC HTB-36), moreover with
a very high efficacy (more than 90% of the transfected cells are analyzable);
an excellent specific signal/background noise ratio; and
a signal of the marker, for example a fluorescent signal that is strong and localized, and therefore perfectly recognizable with the tools that are familiar to a person skilled in the art, for example video-microscopy.

Thus, according to a first aspect, the present invention relates to a method of visualizing the biogenesis of at least one microRNA, preferably a group of microRNAs, in a cell, wherein said method comprises the following steps:
i) transformation of said cell by a vector coding for a protein selected from DGCR8 (DiGeorge syndrome critical region gene 8) protein, Drosha protein, and derivatives thereof, said protein being coupled to a marker;
ii) expression of said protein coupled to said marker; and
iii) detection of said marker.

This step (iii) permits visualization of the biogenesis of said at least one microRNA, preferably of said group of microRNAs, said protein being selected from the DGCR8 protein, the Drosha protein, and derivatives thereof that bind to pri-miRNAs in the course of formation in the vicinity of the transcription sites.

Preferably, the method according to the invention is characterized in that said protein is the DGCR8 protein or a derivative thereof selected from the group consisting of the isoform CRA_a from Homo sapiens (accession number EAX02998 or NP_{—}073557, SEQ ID No.1), the isoform CRA_b from Homo sapiens (accession number EAX02999, SEQ ID No.2), the isoform CRA_c from Homo sapiens (accession number

EAX03000, SEQ ID No.3), the isoform CRA_a from Mus musculus (accession number EDK97512 (SEQ ID No.4), EDK97515 (SEQ ID No.5), NP_{—}201581 (SEQ ID No.6)), the isoform CRA_b from Mus musculus (accession number EDK97513, SEQ ID No.7), the isoform CRA_c from Mus musculus (accession number EDK97514, SEQ ID No.8), the isoform CRA_a from Rattus norvegicus (accession number EDL77930 (SEQ ID No.9), EDL77931 (SEQ ID No.10)), the isoform CRA_b from Rattus norvegicus (accession number EDL77932, SEQ ID No.11) and derivatives thereof.

“Derivative” means any protein possessing a percentage identity with the protein to which this term applies of at least 70%, preferably 80%, more preferably 90%, and even more preferably 95%.

“Percentage identity” between two amino acid sequences in the sense of the present invention denotes a percentage of amino acid residues identical between the two sequences to be compared, obtained after best alignment, said percentage being purely statistical and the differences between the two sequences being randomly distributed over their entire length. “Best alignment” or “optimal alignment” means the alignment for which the percentage identity determined as hereunder is highest. Comparisons of sequences between two amino acid sequences are performed conventionally by comparing these sequences after their optimal alignment, said comparison being performed by segment or by “comparison window” for identifying and comparing local regions of sequence similarity. The optimal alignment of the sequences for the comparison can be performed, besides manually, using the local homology algorithm of

Smith and Waterman (1981), using the local homology algorithm of Neddleman and Wunsch (1970), using the similarity search method of Pearson and Lipman (1988), and by means of computer software using these algorithms (GAP, BESTFIT, BLAST P, BLAST N, FASTA and TFASTA in the Wisconsin Genetics Software Package, Genetics Computer Group, 575 Science Dr., Madison, Wis.).

“Transformation” means modification of the genetic inheritance of a cell by introduction of foreign genetic information. This transformation can be effected by electroporation, conjugation, transfection, transduction, fusion of protoplasts, or any other technique known by a person skilled in the art. Preferably, the method according to the invention is characterized in that the transformation step is a transfection step, preferably carried out transiently according to a chemical method with calcium phosphate or via the use of the transfectant lipofectamine 2000, Invitrogen.

“Vector” means a plasmid, a cosmid, a bacteriophage or a virus in which a polynucleotide coding for the DGCR8 protein or a derivative thereof is inserted, said protein being coupled to a marker. The techniques for construction of these vectors and for insertion of polynucleotides in these vectors are known by a person skilled in the art. In general, any vector that is capable of maintaining itself, of self-replicating or propagating in a host organism and notably in order to induce expression of a protein can be used. A person skilled in the art will select the appropriate vectors notably in relation to the host organism to be transformed and in relation to the transformation technique employed. The vectors of the present invention are notably used for transforming a host organism with a view to expression of a protein selected from the DGCR8 protein, the Drosha protein, and derivatives thereof coupled to a marker in the host organism. Preferably, in the context of this invention, the vector coding for said protein and its marker is a plasmid.

“Marker” means any means, biological, chemical or physical, capable of generating a signal that can be detected and if necessary allowing quantification of the expression of a target gene in a cell. Such markers are well known by a person skilled in the art. A nonlimiting list of these markers comprises the enzymes that produce a signal that is detectable for example by colorimetry, fluorescence or luminescence, such as horseradish peroxidase, alkaline phosphatase, beta galactosidase, glucose-6-phosphate dehydrogenase; chromophores such as fluorescent or luminescent compounds or dyes; electron density groupings detectable by electron microscopy or by their electrical properties such as conductivity, by methods of amperometry or voltammetry, or by measurements of impedance; groups detectable by optical methods such as diffraction, surface plasmon resonance, change in contact angle or by physical methods such as atomic force spectroscopy, tunnel effect, etc.; radioactive molecules such as ³²P, ³⁵S or ¹²⁵I. Their composition will depend in particular on the target gene and on the method of detecting the expression of said target gene. The markers according to the invention can also be antibodies, more particularly specific antibodies of the proteins constituting the Microprocessor, or the elements binding thereto. Preferably, the method according to the invention is characterized in that said marker is a fluorescent marker.

“Fluorescent marker” means any colored, natural, artificial or synthetic substance, absorbing light energy (exciting light) and returning it in the form of fluorescent light (emitted light). In the context of the invention, it will be preferable to use green fluorescent protein (GFP), yellow fluorescent protein (YFP), cyan fluorescent protein (CFP), cytochrome b5, or markers of the Alexa Fluor type, such as Alexa Fluor 488 and Alexa Fluor 555.

Cytochrome b5, of red color, possesses an absorption peak at 412 nm.

GFP is a small protein consisting of 238 amino acids, organized in 11 antiparallel beta strands and a central alpha helix. It is called a “beta-can” structure. The N-terminal and C-terminal ends are accessible for fusion with other proteins. Unmodified, so-called wild-type GFP (wGFP) has two excitation maxima. The first has a wavelength of 395 nm (UV light), the second 475 nm (blue light). The maximum emission wavelength is at 504 nm.

CFP is a protein produced from a mutant of the gene coding for the green fluorescent protein. This protein emits fluorescence at a wavelength of 480 nm when it receives light of wavelength 458 nm.

YFP is a protein produced from a mutant of the gene coding for the green fluorescent protein. This protein emits fluorescence at a wavelength of 527 nm, when it is excited by light of wavelength 514 nm.

The markers of the Alexa Fluor type are fluorochromes produced by the company Invitrogen. These fluorochromes can be coupled to primary or secondary antibodies.

Preferably, in the context of this invention, the fluorescent marker is GFP (Green Fluorescent Protein).

Preferably, the method according to the invention is characterized in that said marker is a protein marker.

Preferably, the method according to the invention is characterized in that the protein selected from the DGCR8 protein, the Drosha protein, and derivatives thereof coupled to a marker protein is a fusion protein, preferably a DGCR8-GFP or Drosha-GFP fusion protein and especially preferably, this fusion protein is the DGCR8-GFP fusion protein having the sequence SEQ ID No.12.

“Fusion protein” means a construction that contains several proteins of different origin. This fusion protein is encoded by a nucleic acid obtained by recombinant DNA technologies, well known by a person skilled in the art. In most cases, one of these proteins is the one that we wish to study whereas the other endows it with properties that make it easy to detect. In the context of this invention, the fusion protein consists of a protein and a marker.

“Detection of the marker” means detection of the signals emitted by the marker, for example detection of fluorescence in the case when the marker is a fluorescent marker.

Preferably, the method according to the invention is characterized in that the step of detection of fluorescence is performed by microscopy, advantageously by means of a fluorescence microscope or according to any other technique familiar to a person skilled in the art.

Preferably, the invention can also be applied by means of immunolabeling. “Immunolabeling” means using a specific antibody of the protein of interest for detecting it directly or indirectly.

The invention thus relates to a method of visualizing the biogenesis of at least one microRNA, preferably a group of microRNAs, in a cell, characterized in that it comprises the following steps:
i) incubation of the cell with at least one antibody selected from the antibodies directed against a protein selected from the DGCR8 protein, the Drosha protein and fragments thereof, said antibody being coupled to a marker; and
ii) detection of said marker.

The coupling of the antibody with the marker permits direct detection of the protein whose recruitment we wish to monitor. It is then called direct immunofluorescence.

“DGCR8 fragment” or “Drosha fragment” means respectively a portion of the peptide sequence of said proteins. Typically, a fragment of a protein can represent 1 to 99% of the peptide sequence of said protein to which said expression relates. Preferably, the respective fragments of the DGCR8 and Drosha proteins number between 1 and 100, preferably 1 to 50 amino acids of the respective peptide sequences of DGCR8 and Drosha.

The invention also relates to a method of visualizing the biogenesis of at least one microRNA, preferably a group of microRNAs, in a cell, wherein said method comprises the following steps:
a) incubation of the cell with at least one primary antibody selected from the antibodies directed against a protein selected from the DGCR8 protein, the Drosha protein and fragments thereof; then
b) incubation of the cell with at least one specific secondary antibody of the primary antibody or antibodies from step a); and
c) detection of said marker.

Coupling of the marker to the secondary antibody permits indirect detection of the primary antibody. It is then called indirect immunofluorescence. Indirect immunofluorescence permits amplification of the fluorescence signals and facilitates monitoring of the recruitment of the DGCR8 and/or Drosha proteins, and thus monitoring of the biogenesis of microRNAs.

Preferably, the primary antibody directed against DGCR8 (or anti-DGCR8 antibody) is a goat antibody. An antibody of this kind is marketed by the company Santa Cruz, under the designation “Sc-48473”.

Preferably, the primary antibody directed against Drosha (or anti-Drosha antibody) is a rabbit antibody. An antibody of this kind is marketed by the company Abcam, under the designation “ab12286”.

Preferably, the labeled secondary antibody directed against the anti-DGCR8 primary antibody is a donkey antibody. Antibodies of this kind are marketed by the company Invitrogen under the designations Invitrogen A11055 and Invitrogen A21432. The Invitrogen A11055 antibody is coupled to a marker of the Alexa Fluor 488 type. This marker is excited at a wavelength of about 495 nm and emits at a wavelength of about 519 nm. The Invitrogen A21432 antibody is coupled to a marker of the Alexa Fluor 555 type. This marker is excited at a wavelength of about 555 nm and emits at a wavelength of about 565 nm.

Preferably, the labeled secondary antibody directed against the anti-Drosha primary antibody is a donkey antibody. Antibodies of this kind are marketed by the company Invitrogen under the designations Invitrogen A21206 and Invitrogen A31572. The Invitrogen A21206 antibody is coupled to a marker of the Alexa Fluor 488 type whereas the Invitrogen A31572 antibody is coupled to a marker of the Alexa Fluor 555 type.

The present invention thus makes it possible to monitor the recruitment of the DGCR8 proteins and/or of the Drosha proteins. Thus, in a particular embodiment, the invention makes it possible to monitor the simultaneous recruitment of these two proteins. If the method of the invention is applied with the antibodies Invitrogen A11055 and Invitrogen A31572, the DGCR8 protein will be visible at a wavelength of about 519 nm whereas the Drosha protein will be visible at a wavelength of about 565 nm.

Alternatively, the antibodies can be labeled with fluorescent markers. In another embodiment, the antibodies can be labeled with markers of the enzymatic type, such as the streptavidin-biotin system. Preferably, said cell is cultivated on a support, fixed and permeabilized before any incubation according to steps i) or a).

Preferably, said cell is cultivated on a suitable support, such as glass slides. Said cell is then fixed on said support according to the conventional techniques of the prior art. Typically, this fixation can be effected by means of aldehyde fixatives such as glutaraldehyde or paraformaldehyde (PFA) or coagulating fixatives, such as methanol, ethanol or acetone. Preferably, said fixation is effected by means of paraformaldehyde. Formaldehyde acts on the —NH₂ groups of the proteins. In this connection, its action leads to crosslinking of the proteins of the membrane and fixation of the cells. Said fixation can be followed by a washing step, preferably washing 3 times with PBS for a time of about 5 minutes. A person skilled in the art will take care to employ suitable conditions permitting conservation of the cellular structures and of the nature of the treated cell. He will thus take care to use solutions having a suitable osmolality and ionic concentration.

Preferably, said cell also undergoes a step of permeabilization, prior to step i) or a) of incubation. The conditions for application of said permeabilization are well known by a person skilled in the art. Typically, it is effected by means of detergents, such as Triton, saponin, sodium dodecylsulfate (SDS), Tween, digitonin, or sodium borohydride. Preferably, permeabilization is effected by treatment with Triton, preferably with TritonX-100 or by treatment with ethanol, preferably 70% ethanol at a temperature of 4° C. for at least 12 h. Typically, the fixative is present at a concentration between 0.1 and 1% relative to the volume of buffer, preferably at a concentration of about 0.4%.

Optionally, said cell can then undergo a step of saturation of the nonspecific binding sites. This saturation can be effected with bovine serum albumin (BSA), or else with serum from the same species as the primary antibody or the secondary antibody. Preferably, this saturation is effected with BSA.

Preferably, after each step of incubation with an antibody, said cell undergoes a washing step. This washing can be effected according to the operating conditions that are well known by a person skilled in the art. Preferably, it comprises 3 washings with phosphate-buffered saline (PBS) for about 5 minutes.

Preferably, for application of the method of the invention, the antibodies are coupled to fluorescent markers. In this case, detection of the marker according to step ii) or c) comprises mounting said cell in a mounting medium suitable for detection of the fluorescent marker. This mounting medium is a suitable medium allowing the fluorescence of the markers to be preserved. The conditions for detection of the marker depend on the nature of said marker and are well known by a person skilled in the art. Typically, said detection is effected by microscopy. Typically, the medium for mounting said cell is a medium comprising Moviol. This mounting medium can also comprise 4,6-diamidino-2-phenylindole (DAPI). The presence of DAPI in the mounting medium permits visualization of the nuclear DNA. The presence of DAPI therefore allows the cell nucleus to be visualized.

In a particular embodiment, the method of the invention can further comprise the detection of the transcription sites according to the conventional techniques of the prior art such as in-situ hybridization (RNA FISH). The method according to the invention therefore permits the simultaneous detection of the transcription sites of said cell (pri-C19MC nascent transcripts) and the Microprocessor (Drosha and DGCR8), thus facilitating monitoring of the biogenesis of the pri-microRNAs in said cell.

The invention is very advantageous since it permits preservation of the DGCR8 and Drosha proteins while optimizing the labeling and monitoring of the biogenesis of microRNAs.

Preferably, the method according to the invention is characterized in that said cell is a placental cell, preferably a placental cell of primates, even more preferably a human placental cell.

Preferably, the method according to the invention is characterized in that said placental cell was derived from a choriocarcinoma, preferably said cell is selected from the group comprising the human choriocarcinoma lines JEG 3 (ATCC HTB-36), JAR (ATCC HTB-144) and BeWo (ATCC CCL-98). More preferably, said cell was derived from the human choriocarcinoma line JEG 3 (ATCC HTB-36). This immortalized line is easy to manipulate and cultivate in DMEM (4.5 g/ml glucose, 5% calf serum under penicillin/streptomycin antibiotics) and expresses the C19MC locus in a strong, constitutive and monoallelic manner.

The C19MC chromosomal locus is a gene locus, positioned on human chromosome 19 and containing 46 genes of microRNAs organized in tandem and extending to about 100 kb. The genes of these microRNAs are expressed mainly, or even exclusively, in the placenta.

Thus, preferably, the method according to the invention is characterized in that said at least one microRNA, preferably said group of microRNAs, is encoded by the C19MC chromosomal locus (chromosome 19 miRNA cluster), preferably said miRNA is selected from the group comprising SEQ ID No.13 to SEQ ID No.58.

Preferably, the method according to the invention is characterized in that it is intended for identifying compounds capable of modulating the expression of at least one microRNA, preferably a group of microRNAs, and in that it further comprises the steps of:
1) bringing said cell into contact with a test compound,
2) measuring the expression of said marker in the presence of and in the absence of said test compound, and
3) selection of the compound or compounds permitting the induction of a decrease or an increase, preferably a decrease, in the expression of the marker.

Thus, the invention relates to a method of screening of compounds capable of modulating the expression of at least one microRNA, preferably a group of microRNAs, in a cell, wherein said method comprises the following steps:
transformation of said cell by a vector coding for a protein selected from the DGCR8 protein, the Drosha protein, and derivatives thereof, said protein being coupled to a marker;
expression of said protein coupled to said marker;
bringing said cell into contact with a test compound;
measuring the expression of said marker in the presence of and in the absence of said test compound; and
selection of the compound or compounds permitting the induction of a decrease or an increase, preferably a decrease, in the expression of the marker.

The present invention relates to a method of screening of compounds capable of modulating the expression of at least one microRNA, preferably a group of microRNAs, in a cell, characterized in that it comprises the following steps:
1) bringing said cell into contact with a test compound;
2) incubation of the cell with at least one antibody selected from the antibodies directed against a protein selected from the DGCR8 protein, the Drosha protein and fragments thereof, said at least one antibody being coupled to a marker;
3) detection of said marker in the presence of and in the absence of said test compound; and
4) selection of the compound or compounds permitting the induction of a decrease or an increase, preferably a decrease, in the expression of the marker.

The present invention also relates to a method of screening of compounds capable of modulating the expression of at least one microRNA, preferably a group of microRNAs, in a cell, characterized in that it comprises the following steps:
A) bringing said cell into contact with a test compound;
B) incubation of the cell with at least one primary antibody selected from the antibodies directed against a protein selected from the DGCR8 protein, the Drosha protein and fragments thereof;
C) incubation of the cell with at least one specific secondary antibody of the primary antibody or antibodies from step B);
D) detection of said marker in the presence of and in the absence of said test compound; and
E) selection of the compound or compounds permitting the induction of a decrease or an increase, preferably a decrease, in the expression of the marker.

The purpose of the method of screening of molecules according to the invention is to identify molecules or cellular factors capable of modulating the action of the Microprocessor in the nucleoplasm and therefore the expression of the microRNAs, in particular by inhibiting the recruitment of the DGCR8 protein or of the Drosha protein.

This method in fact makes it possible to screen various molecules having an influence on the recruitment of the DGCR8 protein only, the Drosha protein only, or else of both proteins DGCR8 and Drosha. As deregulations of the production of the microRNAs are involved in pathological contexts such as cancer, this method of screening according to the invention moreover makes it possible to identify molecules or cellular factors involved in said pathological contexts. The compounds to be tested according to the method of screening according to the invention can be of lipid, carbohydrate, protein or nucleic acid nature. Preferably, these compounds are siRNAs, more preferably siRNAs of the human pangenomic siRNA database. In particular, the test compounds can be described in artificial or natural libraries of chemicals.

“Measurement of expression” means the localization and qualitative and/or quantitative measurement of the marker used, in particular in the case of a fluorescent marker, measurement of fluorescence, applied by techniques known by a person skilled in the art. Thus, for example, in the method according to the present invention, automated microscopy can be used for qualitative measurement of the presence or absence of the fluorescent marker used (for example GFP) in said cell at the transcription site.

### LEGENDS OF FIGURES

FIG. 1: Schematic representation of the chromatin C19MC locus (˜100 kb). This figure shows the genetic organization of the miRNAs by revealing the repeated character of this locus: the majority of the genes of the miRNAs are contained in introns, which in their turn are localized within a noncoding sequence (400-700 nt) repeated in direct tandem. The stem-loop structure represents a gene of miRNA (pre-miRNA) and oligonucleotide probes used for the fluorescent in situ hybridization experiments (RNA

FISH) are symbolized by gray rectangles.

FIG. 2: Visualization by RNA FISH of the nascent pri-miRNAs in JEG3 choriocarcinomas. The JEG3 cells are hybridized to oligonucleotide probes coupled to Cy3 (a and d) or Alexa Fluor 488 (b and e) that reveal intron or exon sequences of the pri-miRNA as indicated. In the majority of the nuclei, a single RNA signal is observed after superposition of these signals (c and f) which, in 50% of cases, has a characteristic doublet or dumb-bell shape: these are the immature pri-miRNAs in the vicinity of the transcription site (FIG. 1D).

FIG. 3: The histograms show the proportion of nuclei with 0, 1, 2, or 3 pri-miRNA signals (more than 300 nuclei analyzed).

FIG. 4: Drosha and DGCR8 are concentrated at the C19MC locus by immunofluorescence. The JEG3 cells are hybridized to probes revealing the pri-miRNA before being immunolabeled with antibodies directed against the endogenous proteins Drosha (top) and DGCR8 (bottom).

FIG. 5: Effect of the loss of function of DGCR8 on recruitment of Drosha. The JEG3 cells are transfected by an siRNA against DGCR8 (or a control siRNA) and then are hybridized to an oligo probe detecting the pri-miRNA (b and e) before being immunolabeled with an antibody against Drosha (a and d). The superpositions of the DGCR8 and pri-miRNA signals are shown (c and f).

FIG. 6: The histograms show the proportion of nuclei with a Drosha signal (left) or DGCR8 signal (right) in the cells transfected with siRNAs against the mRNAs of DGCR8 or Drosha. The value observed in the controls cells is set at 100%.

FIG. 7: Visualization of the recruitment of GFP-DGCR8 at the C19MC locus on immobilized cells. A eukaryotic vector expressing a GFP-DGCR8 fusion (GFP-DGCR8) is transfected transiently in the JEG3 cells which are then hybridized to oligos that reveal the pri-miRNAs (pri-miRNA). Top: schematic representation of the GFP-DGCR8 fusion with notably the functional protein domains of DGCR8.

FIG. 8: Visualization of the recruitment of GFP-Drosha at the C19MC locus on immobilized cells. A eukaryotic vector expressing a GFP-DROSHA fusion is transfected transiently in the JEG3 cells which are then hybridized to oligos that reveal the pri-miRNAs (pri-miRNA). Top: schematic representation of the GFP-Drosha fusion with notably functional protein domains of Drosha.

FIG. 9: Simultaneous visualization in the JEG3 choriocarcinomas:
of nascent pri-miRNAs by RNA FISH at the C19MC locus,
of recruitment of the DGCR8 protein; and
of recruitment of the Drosha protein.

FIG. 10: Automated visualization of the DGCR8 protein at the C19MC locus (96-well plates)
A) Detection of the IF-DGCR8 signals in the vast majority of the nuclei (a field of nuclei (visualized by DAPI labeling) selected at random is shown)
B) Automated recognition of the IF-DGCR8 signals by means of imaging software.

### EXAMPLES

### Example 1

### The biogenesis of the microRNAs

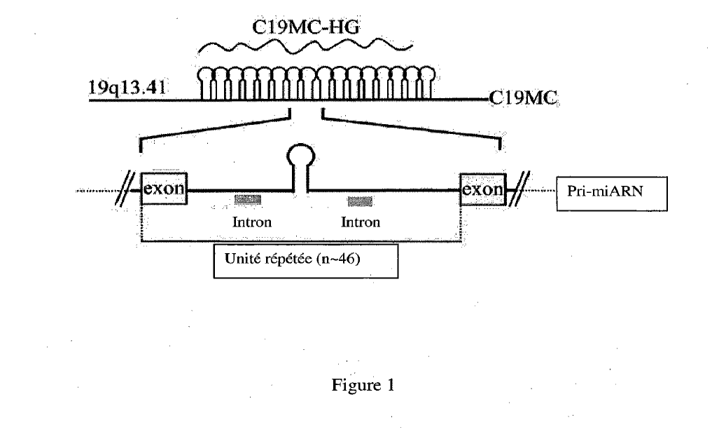

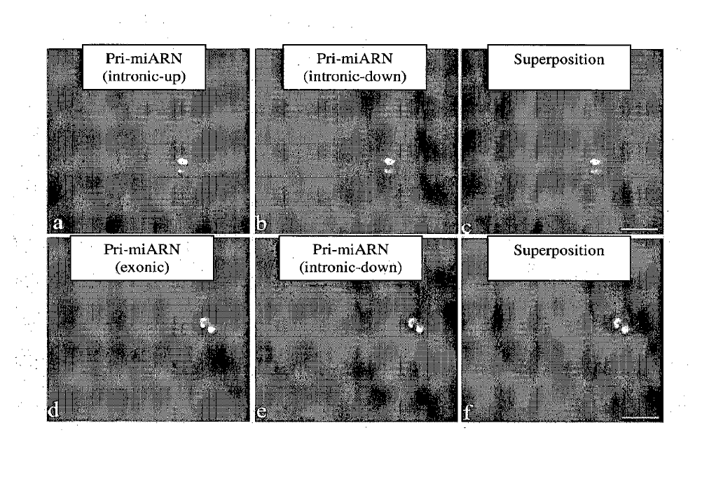

To throw light on the organization of the biogenesis of microRNAs (or miRNAs), the inventors employed a cell imaging approach enabling them to monitor the intranuclear fate of the transcripts of pri-miRNAs in cells in culture, fixed or living. To do this, the inventors used the chromosomal locus positioned on chromosome 19 (chromosome domain 19q13), which contains 46 genes of microRNAs: it is the C19MC locus. Most of the genes of pri-miRNAs of C19MC are integrated in highly repetitive sequences with a length of 400 to 700 nucleotides (FIG. 1). The inventors exploited the repetitive nature of C19MC-HG (C19MC Host Gene) for performing a fluorescent in-situ hybridization (method called “RNA FISH”) by means of oligonucleotide probes capable of hybridizing to repetitive intron and exon sequences of C19MC-HG (FIG. 2). By using a cell line of choriocarcinomas of the JEG3 type as well as two different DNA probes, hybridizing respectively to sequences upstream and downstream of the pri-miRNAs, the inventors identified important signals corresponding to the nuclear pri-miRNAs, moreover in the majority of the cells (data not shown).

The inventors found that about 50% of these pri-RNA signals are visualized in the form of a doublet (FIG. 2 a,b,c) or in an even more complex form that can extend to several μm of length and occupy an important place in the nucleus (not shown). These RNA signals correspond to the non-spliced (or partially spliced) pri-miRNA transcripts, and not to the spliced introns detached from the pri-miRNAs since they are also revealed with exon probes (FIG. 2, d,e,f). These signals were detected near one of the three DNA signals revealed by DNA FISH (data not shown), which indicates that they represent transcripts freshly obtained in the vicinity of the transcription sites (note: the JEG3s are triploid).

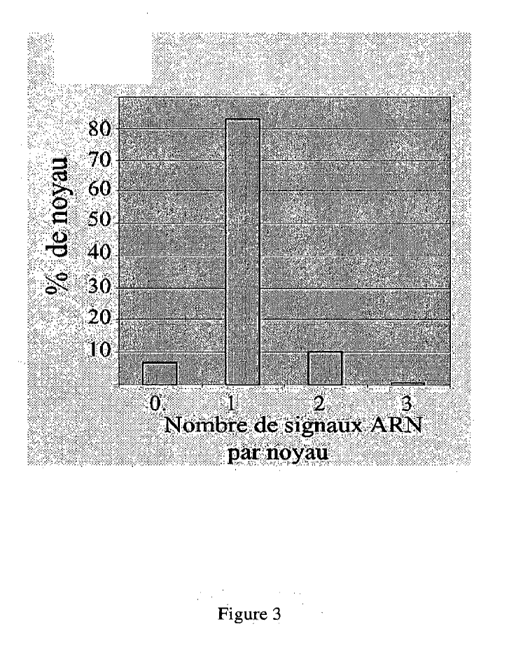

Using the intron and exon probes, the inventors also identified many signals in the form of points completely encircling the intron RNA signals, notably in the vicinity of the transcription site (data not shown). These signals correspond to the pri-miRNAs, which leave their transcription site and traverse the nucleoplasm. These hybridizations by RNA FISH also show that the C19MC chromosomal locus is preferably, or even exclusively, expressed in mono-allelic manner by the human choriocarcinoma line JEG3 (data not shown and FIG. 3).

### Example 2

### The Microprocessor Combines with the Pri-miRNAs

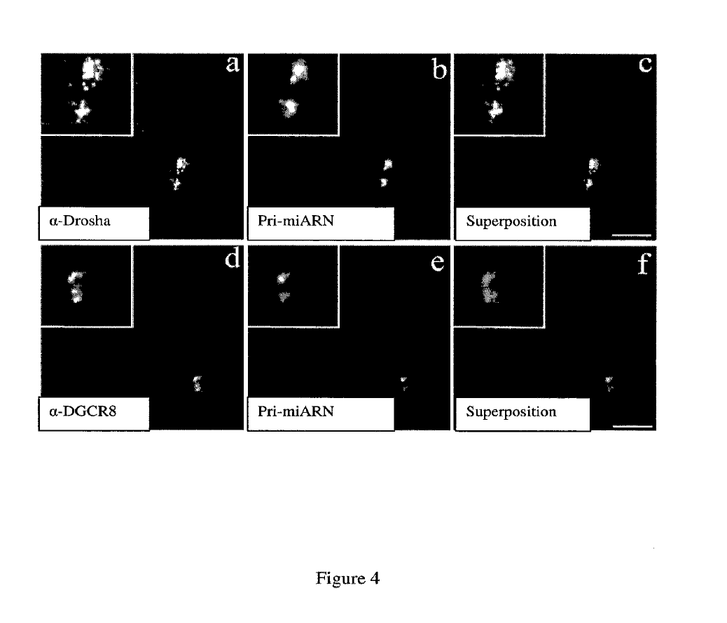

To determine whether the Microprocessor can be visualized on the pri-miRNAs of the freshly synthesized C19MC locus, the inventors combined RNA FISH experiments with experiments for detection of proteins by immunofluorescence, according to the techniques familiar to a person skilled in the art. The inventors were thus able to detect the endogenous Drosha or DGCR8 proteins (FIG. 4). The inventors observed a co-localization of intense immunofluorescence signals for Drosha and

DGCR8 with the signals of the pri-miRNAs, indicating that the Microprocessor combines with the non-spliced pri-miRNAs in the vicinity of the transcription site. This important nuclear accumulation of the Microprocessor has also been demonstrated in two other choriocarcinoma cell lines, JAR and BeWO, which express the genes of the C19MC locus. In contrast, the HeLA and HEK293 cells that do not express the genes localized on the C19MC locus only show very weak signals connected with the Microprocessor (data not shown).

Recruitment depends on transcription and probably occurs via interaction with the RNAs, since there is no detection in cells treated with Actinomycin, in which C19MC-HG is not detected near the transcription site of C19MC (data not shown).

### Example 3

### DGCR8 has a Role in the Recruitment and/or Stabilization of Drosha at the Level of the Pri-miRNAs

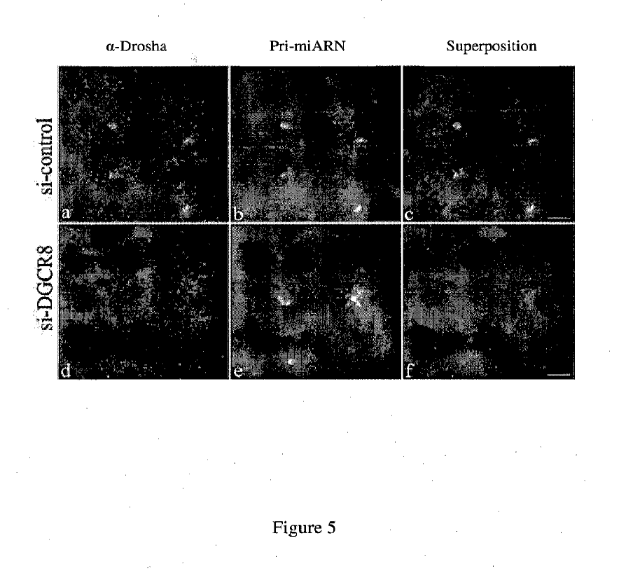

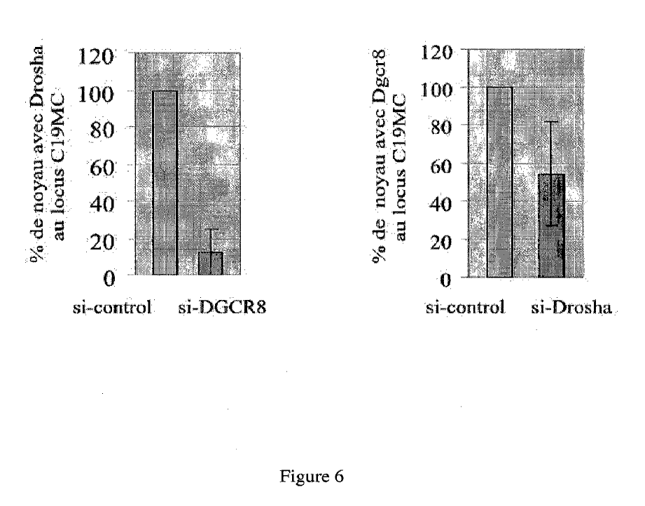

In order to determine whether the recruitment of Drosha and DGCR8 on the C19MC transcripts is interdependent, the expression of these two proteins was decreased by means of RNAi (according to the well-known techniques of “knocking-down”), and the impact of the deficiency of DGCR8 on the intranuclear distribution of Drosha (and vice-versa) was evaluated. The inventors thus validated the test of deficiency of genes by showing that only 20% of the cells derived from the choriocarcinoma line of type JEG3, transformed with siRNAs (small interfering RNAs) directed against mRNAs coding for DGCR8 or Drosha, have detectable immunofluorescence signals of the target proteins at the C19MC locus (data not shown). The proportion of nuclei with a Drosha signal at the C19MC locus is greatly reduced in cells with deficiency of DGCR8 (FIGS. 5 and 6 (left)), which indicates that DGCR8 has a role in the recruitment and/or stabilization of Drosha at the level of the pri-miRNAs. Deficiency of Drosha also has an impact with respect to recruitment of DGCR8, even if a substantial fraction of the nucleus still has DGCR8 proteins at the C19MC locus (data not shown and FIG. 6 (right)). The inventors also found that in the absence of Drosha, DGCR8 is redistributed in the nucleus, as well as in the nucleolus (data not shown).

### Example 4

### Visualization of the Recruitment of GFP-DGCR8 in Living Cells

In order to elucidate the mechanism by which the Microprocessor is directed to the transcripts of the C19MC locus, JEG3 cells were transfected by the plasmid coding for the DGCR8 protein, coupled to GFP according to techniques familiar to a person skilled in the art.

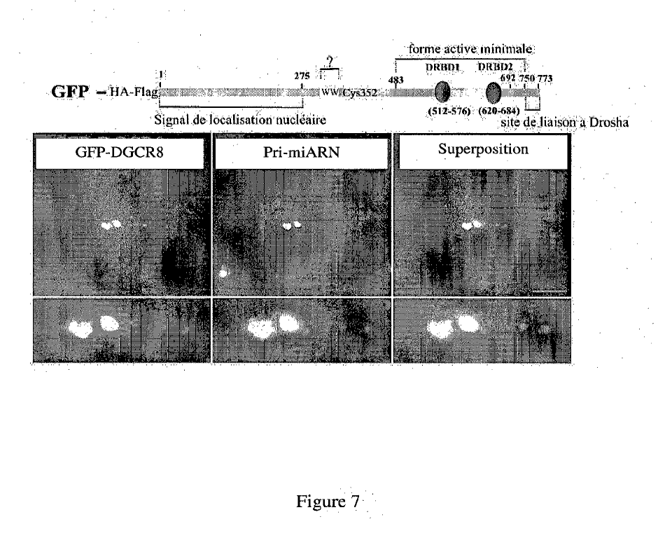

The inventors showed that the DGCR8 protein coupled to GFP concentrates at the C19MC locus (FIG. 7). Signals in the form of a doublet at the level of C19MC are also visible on living cells, which indicates that these signals are not artifacts connected with the fixation and/or hybridization steps. Moreover, the signals detected that are connected with the GFP marker are co-localized with the RNA signals around the transcription sites, but also with the signals observed throughout the nucleus. This suggests that the pri-miRNAs may be present at localizations other than their transcription sites.

### Example 5

### Visualization of the Recruitment of GFP-Drosha in Living Cells

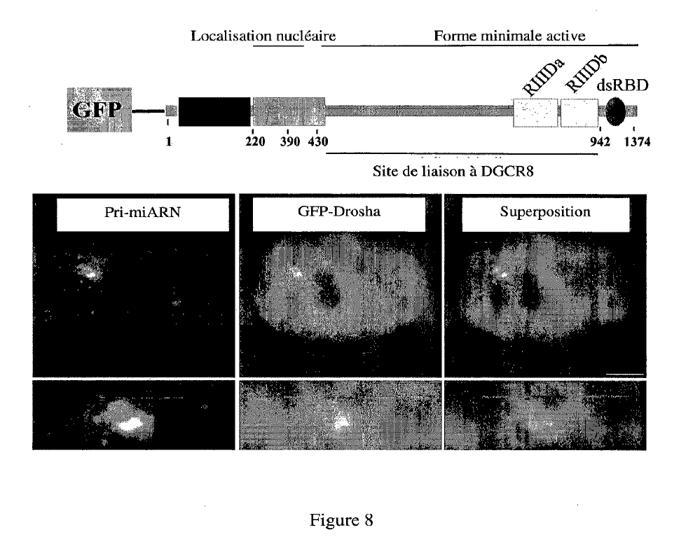

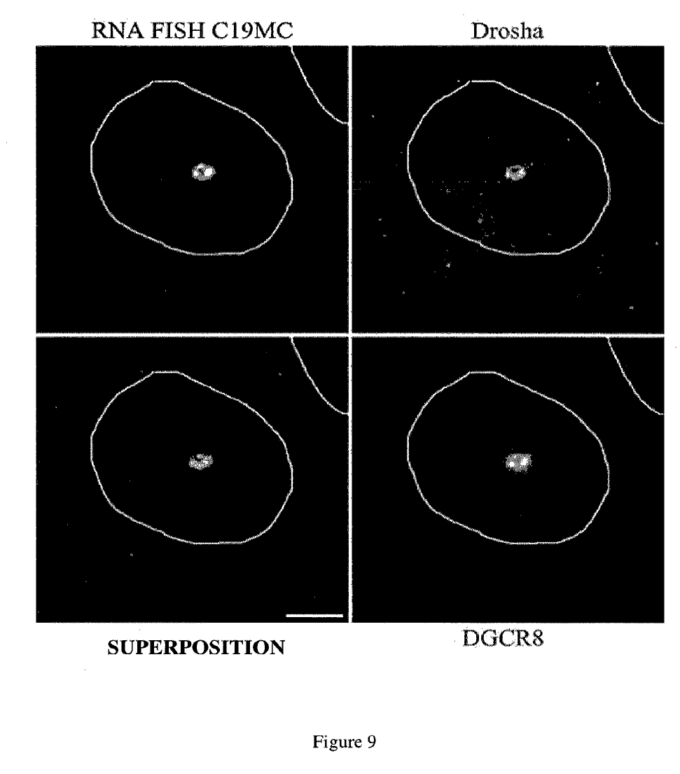

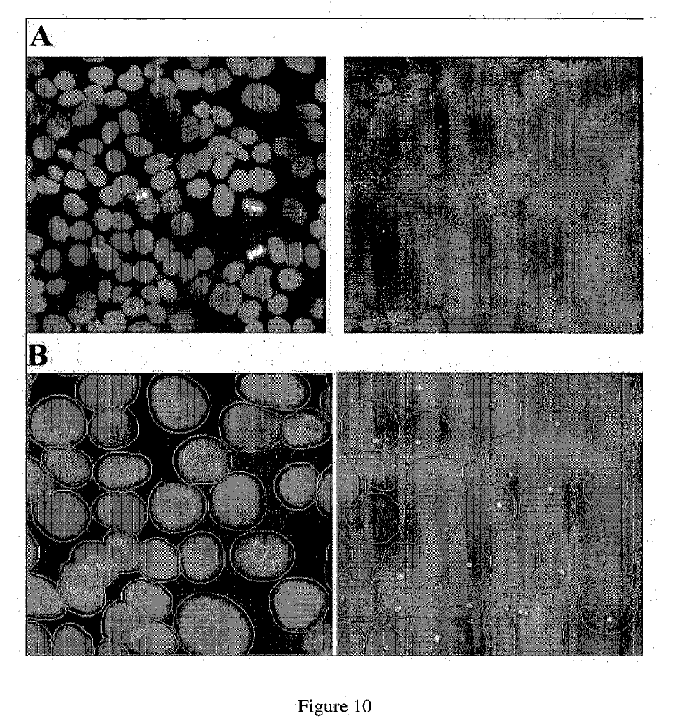

As described in example 4, JEG3 cells were transfected by the plasmid coding for the Drosha protein, coupled to GFP according to techniques familiar to a person skilled in the art. The inventors have thus shown that the Drosha protein coupled to GFP also concentrates at the C19MC locus (FIG. 8).

### Example 6

### The Proteins Associated with the Microprocessor

DGCR8 and Drosha are necessary and sufficient to permit maturation of the pri-miRNAs. However, a proteomic analysis identified other proteins associated with the Microprocessor, for example RNA helicases, heterogeneous nuclear ribonucleoproteins (hnRNP) and other proteins having an RNA binding motif. The techniques employed for visualizing the biogenesis of the pri-miRNAs at the C19MC locus enabled the inventors to determine the presence of other RNA-binding proteins. Thus, the inventors showed that the presence of C1/C2 hnRNP, EWS, ILF3/NFAR/NF90 and RNA helicase A (RHA) at the C19MC locus (data not shown), which indicates that these proteins probably play a role in the synthesis, intranuclear organization and maturation of the pri-miRNAs encoded by the C19MC locus. The recruitment of the C1/C2 hnRNPs is specific since the Al hnRNPs and other hnRNPs analyzed, such as the M1M2, U and A2/B1 hnRNPs, are not found to be concentrated at the C19MC locus. The inventors also showed that nucleolin, the Al hnRNPs, the RNA helicases p68 and 672, or the exosomes (PSsc100), are not found to be concentrated significantly at the C19MC locus (data not shown).

### Example 7

### Detection of the Biogenesis of microRNAs by Immunofluorescence Directed Against the Proteins of the Microprocessor

### Protocol

JEG3 cells are cultivated on glass slides previously treated with 1% gelatin. These cells are fixed for 15 minutes at room temperature with 4% paraformaldehyde in phosphate-buffered saline (PBS). The cells fixed in this way then undergo a step of permeabilization for 5 to 7 minutes at a temperature of 4° C. in a medium consisting of concentrated Triton X-100 at 0.4% in PBS.

The cells are then incubated in a humid chamber, successively with the anti-Drosha and anti-DGCR8 primary antibodies in a PBS1X/1%BSA medium as described below:
the cells are incubated with an anti-DGCR8 primary antibody at a dilution of 1/300 and at room temperature. An antibody of this kind is marketed by Santa Cruz under the designation “sc-48473”.
the cells are also incubated with an anti-Drosha primary antibody at a dilution of 1/300 and at a temperature of 37° C. An antibody of this kind is marketed by Abcam under the designation “ab12286”.

The cells then undergo 3 washings of 10 minutes in PBS at room temperature.

The cells are then incubated with the secondary antibodies directed respectively against the anti-DGCR8 and anti-Drosha antibodies as described below:
the cells are incubated with an anti-DGCR8 antibody at a dilution of about 1/500 and at room temperature. Antibodies of this kind are marketed by the company Invitrogen under references A11055 and A21432;
the cells are also incubated with anti-Drosha antibodies at a dilution of 1/500 and at a temperature of 37° C. Antibodies of this kind are marketed by the company Invitrogen under references A21206, or A31572.

The cells then undergo 3 washings of 10 minutes in PBS at room temperature.

The slides are then mounted with Moviol/DAPI at a concentration of 0.1 μg/ml. The presence of DAPI in the mounting medium permits visualization of the nuclear DNA.

### Result

As shown in FIG. 9, the inventors successfully used a technique for simultaneous visualization of the transcription sites of the cells treated according to the method of the invention, the recruitment of DGCR8 and the recruitment of the Drosha protein. The superposition of the signals provides evidence of the relevance of the method for monitoring the biogenesis of microRNAs.

### Example 8

### Automated Monitoring of the Biogenesis of microRNAs

The inventors used the protocol as defined in example 6 for cultivating JEG3 cells on 96-well plates, which are used conventionally when developing strategies for high-throughput screening. As shown by part A of FIG. 9, the great majority of the nuclei (here revealed by labeling with DAPI) display obvious immunofluorescence (IF) (illustrated here by the detection of DGCR8). Remarkably, the signal/noise ratio is such that it permits automated, highly specific recognition by imaging software (part B of FIG. 9). FIG. 10 illustrates detection of the DGCR8 protein according to this protocol and by means of said software.

1. A method of visualizing the biogenesis of at least one microRNA, preferably a group of microRNAs, in a cell, wherein said method comprises the following steps:
i) incubation of the cell with at least one antibody selected from the antibodies directed against a protein selected from DGCR8 (DiGeorge syndrome critical region gene 8) protein, Drosha protein and fragments thereof, said antibody being coupled to a marker; and
ii) detection of said marker.

2. A method of visualizing the biogenesis of at least one microRNA, preferably a group of microRNAs, in a cell, wherein said method comprises the following steps:
a) incubation of the cell with at least one primary antibody selected from the antibodies directed against a protein selected from the DGCR8 protein, the Drosha protein and fragments thereof; then
b) incubation of the cell with at least one specific secondary antibody of the primary antibody or antibodies from step a); and
c) detection of said marker.

3. A method of visualizing the biogenesis of at least one microRNA, preferably a group of microRNAs, in a cell, wherein said method comprises the following steps:
transformation of said cell by a vector coding for a protein selected from the DGCR8 protein, the Drosha protein, and derivatives thereof, said protein being coupled to a marker;
expression of said protein coupled to said marker; and detection of said marker.

4. The method as claimed claim 1, wherein said protein is the DGCR8 protein or a derivative thereof selected from the group consisting of the isoform CRA_a from Homo sapiens (accession number EAX02998 or NP_{—}073557), the isoform CRA_b from Homo sapiens (accession number EAX02999), the isoform CRA_c from Homo sapiens (accession number EAX03000), the isoform CRA_a from Mus musculus (accession number EDK97512, EDK97515, NP_{—}201581), the isoform CRA_b from Mus musculus (accession number EDK97513), the isoform CRA_c from Mus musculus (accession number EDK97514), the isoform CRA_a from Rattus norvegicus (accession number EDL77930, EDL77931), the isoform CRA_b from Rattus norvegicus (accession number EDL77932) and derivatives thereof.

5. The method as claimed in claim 3, wherein said marker is a fluorescent marker, preferably said fluorescent marker is GFP (Green Fluorescent Protein).

6. The method as claimed in claim 1, wherein said marker is a protein marker.

7. The method as claimed in claim 6, wherein said protein coupled to a protein marker is a fusion protein, preferably a DGCR8-GFP or Drosha-GFP fusion protein and especially preferably the DGCR8-GFP fusion protein having the sequence SEQ ID No.12.

8. The method as claimed in claim 1, wherein said step of fluorescence detection is performed by microscopy.

9. The method as claimed in claim 1 wherein said cell is a placental cell, preferably a human placental cell.

10. The method as claimed in claim 9, wherein said placental cell is a cell obtained from a choriocarcinoma, preferably said cell is selected from the group comprising the human choriocarcinoma lines JEG 3 (ATCC HTB-36), JAR (ATCC HTB-144) and BeWo (ATCC CCL-98).

11. The method as claimed in claim 1, wherein said microRNA is encoded by the C19MC chromosomal locus (chromosome 19 miRNA cluster), preferably said microRNA is selected from the group comprising the sequences SEQ ID No.13 to 58.

12. A method of screening compounds capable of modulating the expression of at least one microRNA, preferably a group of microRNAs, in a cell, wherein said method comprises the following steps:
transformation of said cell by a vector coding for a protein selected from the DGCR8 protein, the Drosha protein, and derivatives thereof, said protein being coupled to a marker;
expression of said protein coupled to said marker;
bringing said cell into contact with a test compound;
measuring the expression of said marker in the presence of and in the absence of said test compound; and
selecting the compound or compounds permitting the induction of a decrease or an increase, preferably a decrease, in the expression of said marker.

13. A method of screening compounds capable of modulating the expression of at least one microRNA, preferably a group of microRNAs, in a cell, wherein said method comprises the following steps:
1) bringing said cell into contact with a test compound;
2) incubating the cell with at least one antibody selected from the antibodies directed against a protein selected from the DGCR8 protein, the Drosha protein and fragments thereof, said antibody being coupled to a marker;
3) detecting said marker in the presence of and in the absence of said test compound; and
4) selecting the compound or compounds permitting the induction of a decrease or an increase, preferably a decrease, in the expression of the marker.

14. A method of screening compounds capable of modulating the expression of at least one microRNA, preferably a group of microRNAs, in a cell, wherein said method comprises the following steps:
A) bringing said cell into contact with a test compound;
B) incubating the cell with at least one primary antibody selected from the antibodies directed against a protein selected from the DGCR8 protein, the Drosha protein and fragments thereof; then
C) incubating the cell with at least one specific secondary antibody of the primary antibody or antibodies from step B);
D) detecting said marker in the presence of and in the absence of said test compound; and
E) selecting the compound or compounds permitting the induction of a decrease or an increase, preferably a decrease, in the expression of the marker.

15. The method of screening as claimed in claim 12, wherein said test compounds are siRNAs.

16. The method of screening as claimed in claim 13, wherein said test compounds are siRNAs.

17. The method of screening as claimed in claim 14, wherein said test compounds are siRNAs
